(19) United States
(12) Patent Application Publication
(10) Pub. No.: US 2006/0195343 A1
(43) Pub. Date: Aug. 31, 2006

(54) Method and program-encoded medium for computerized synchronization of distributed radiology system

(76) Inventor: Karol Ruckschloss, Erlangen (DE)

Correspondence Address: SCHIFF HARDIN, LLP;PATENT DEPARTMENT, 6600 SEARS TOWER, CHICAGO, IL, 60606-6473, US

(21) Appl. No.: 11/344,818

(22) Filed: Jan. 31, 2006

(30) Foreign Application Priority Data
Jan. 31, 2005 (DE) 10 2005 004 470.0

(51) Int. Cl. G06F 19/00; A61B 5/00

(52) U.S. Cl. 705/3

(57) ABSTRACT

Inconsistencies between the local data sets of a distributed radiology system are detected and corrected centrally. The corrections are communicated to the affected actors of the radiology system. The data sets of the affected actors are synchronized by incorporation of the corrections. Wrong associations and losses of x-ray images are thereby prevented.

### BACKGROUND OF THE INVENTION

1. Field of the Invention

The present invention is directed to a computerized method for synchronizing a distributed radiology system, as well as a computer-readable medium encoded with a computer program for implementing such a method.

2. Description of the Prior Art

Among other things, cooperation (collaboration) in a distributed radiology system is regulated in the international standard IHE (Integrating Healthcare Enterprise). The standard IHE starts with the standards DICOM (Digital Imaging and Communications in Medicine) and HL7 (Health Level 7) and extends these with regard to the profiles or that are relevant to the IHE standard. To describe the cooperation, actors, their roles as well as their participation in the scenarios are defined in the IHE standard.

For example, the following actors and roles are defined in the IHE standard.
Order Filler—is typically formed by what is known as a Radiology Information System (RIS). The RIS serves for registration of patients and for planning of examinations. Changes to the associated data are implemented at the RIS and forwarded from there to other components of the distributed system.
Image Manager/Image Archive—is typically formed by what is known as a Picture Archiving and Communication System (PACS). Images are stored and provided here. Furthermore, notification about implemented examination steps is made.
Acquisition Modality: Images are acquired and sent to the Image Manager in this actor.

These actors are participating, for example, in the following scenarios:
Scheduled Workflow: The Scheduled Workflow is the standard scenario for the cooperation in a distributed radiology system. Examinations are initially properly planned at the RIS. The patient and examination data thereby generated are send to the Acquisition Modality in the form of a Modality Worklist and to the PACS in the form of an Order Schedule, such that all participating components of the system exhibit a concurrent, consistent (i.e. synchronous) data set. Images are acquired at the Acquisition Modality at a later point in time, associated with the locally-stored data and sent to the PACS together with key data characterizing them. At the PACS, the images are stored and associated with the locally-stored patient and examination data with the aid of the received characterizing key data. The local associations are implemented identically in all components of the system because their local data sets exhibit synchronous key data due to the defined workflow.
Patient Information Reconciliation: This scenario occurs, for example, after an emergency acquisition. The images are acquired first. The necessary patient and examination data are also input by hand at the Acquisition Modality. The images and, if applicable, also the acquired data are sent to the PACS. Data inconsistencies between RIS and PACS are avoided via a defined message exchange.

As a consequence of the standardized interfaces and workflows, the actors can easily be realized and marketed not only as an integrated product, but also as a system composed of multiple products separately realized and sold by different manufacturers that respectively implement a part of the standardized overall functionality and interact such that overall the same functionality is realized as given an integrated realization of the standardized functioning.

These products also can be fashioned as computer program products that are executed by hardware (for example at least one processor) by which the visible, objective execution environment of the products is formed. This execution is frequently supported by support software (for example multitasking or, respectively, multithreading operating system, databank system, Windows system).

The realization of the above for specific solutions is a complex technical problem as a consequence of the distribution of the system and the number of different system components and requirements.

### SUMMARY OF THE INVENTION

It is an object of the present invention to recognize at least one of the existing problems associated with synchronization of a distributed radiology system and to solve this problem by specification of at least one technical action.

The invention is based on the following realizations:
Under the data there are two key attributes that are not considered in the standard HL7:
   Accession Number (AccNo) that serves for identification of an Order/Imaging Service Request;
   Study Instance UID (SIUid) with whose help a Requested Procedure is identified.
There are modalities that do not have a Modality Worklist interface. In such modalities all patient data and examination data are manually entered and not automatically imported via a worklist. Among other things, the Accession Number or the Study Instance UID is thereby also recorded. Wrong (i.e. asynchronous) data can hereby be created, for example as a result of typing errors.
There are products in which a manual change of the local data is possible. Due to a local change of an Accession Number or a Study Instance UID, subsequently false (i.e. not synchronous) data can thus be created, even given correct transmission with the aid of a Modality Worklist.
The Order Filler RIS and the Image Manager PACS are real, frequently separate system components with separate local data sets that are acquired at one of the components and forwarded to the other components (and therewith initially automatically synchronized) via the HL7 messages. The data exchange required for this purpose is defined in the extension IHE standard of the standard HL7 in the previously-described scenarios “Scheduled Workflows” and “Patient Information Reconciliation”. In the IHE standard, it is described how the Accession Number and the Study Instance UID are to be sent in an order message from the Order Filler RIS to the Image Manager PACS.

However, it is unspecified as to how these values should be corrected in the event that they should no longer be synchronous, as indicated in the preceding. The cited scenarios assume that a synchronicity produced once at the beginning will be permanently maintained. No profiles exist as to how incorrect keyed data from examinations should be centrally corrected during the operation and how this correction should be forwarded to other system components so that the data sets of the participating actors are consistent again (i.e. synchronous).
When false data are sent to the PACS with the images, the images can either not be associated at all or it leads to wrong associations. This can lead to repetition of examinations, data loss or misdiagnoses as a consequence of falsely-associated images.

The known techniques do not solve this recognized problem.

The above object is achieved in accordance with the present invention by a method for synchronization of a distributed radiology system having at least two actors, such an order filler and an image manager functioning as an image archive and/or as an image acquisition modality, the system providing for local data retention at the actors, with data representing radiology examination results being stored, for each examination, in at least two data memories, the data being formulated as an accession number or as a study instance, and wherein an inconsistency between the distributed, stored data is detected and displayed, thereby causing the automatic acquisition of corrected data, with the corrected data being communicated to the actors affected by the inconsistent data, and wherein the distributed radiology system is synchronized by correction of the stored data with the communicated, corrected data.

The inventive embodiments of the method can be fashioned as a computer program product (i.e. a computer-readable medium encoded with a computer program), that causes the computer in which it is loaded to implement the inventive method described above with its program code being executed by a processor.

Moreover, individual components of the method described in the preceding can be executed in one commercial unit and the remaining components can be executed in another commercial unit. The invention thus can be embodied in a product that includes:

at least one Order Filler,

at least one Image Manager comprising an Image Archive and/or

at least one Acquisition Modality

of a distributed radiology system which comprises means that are directed towards implementation of those steps of a method according to at least one of the method aspects described in the preceding that are effected by the product, whereby at least one further product is directed towards implementation of the remaining steps of the method, and all steps of the method are implemented via interaction of the at least two products.

### DESCRIPTION OF THE DRAWINGS

FIG. 1 schematically illustrates an exemplary distributed radiology system RS, having a number of actors that are fashioned as an Order Filler RIS, Image Manager PACS and Acquisition Modality AM.

FIG. 2 shows an exemplary data structure for the administration of patient and examination data as well as of examinations (and images generated thereby).

FIG. 3 illustrates correction of an inconsistent data instance AccNo with an unknown data instance AccNo.

FIG. 4 illustrates correction of an inconsistent data instance AccNo with a known data instance AccNo FIG. 5 illustrates correction of an inconsistent data instance SIUid with an unknown data instance SIUid.

FIG. 6 illustrates correction of an inconsistent data instance SIUid with a known data instance SIUid.

FIG. 7 illustrates correction of an inconsistent data instance PatId.

FIG. 8 illustrates correction of inconsistent data instances PatId, SIUid.

FIG. 9 illustrates correction of inconsistent data instances PatId, AccNo.

FIG. 10 illustrates correction of inconsistent data instances PatId, AccNo, SIUid.

### DESCRIPTION OF THE PREFERRED EMBODIMENTS

As a solution for the desired synchronization, a distributed radiology system RS in accordance with the invention detects falsely keyed data, so as to be able to correct it centrally and to forward the corrected data to the actors affected by the detected data inconsistency so that the actor's data sets are consistent (i.e. synchronous) again.

Particularly good advantages result when the standards that already exist (such as, for example, the IHE or the HL7 standards, DICOM) are extended for this purpose. By this extension it is possible to remedy inconsistencies between the local data sets of actors of a distributed radiology system that span across manufacturers. This extension can be applied to all products that function as these actors. They thus can be standardized.

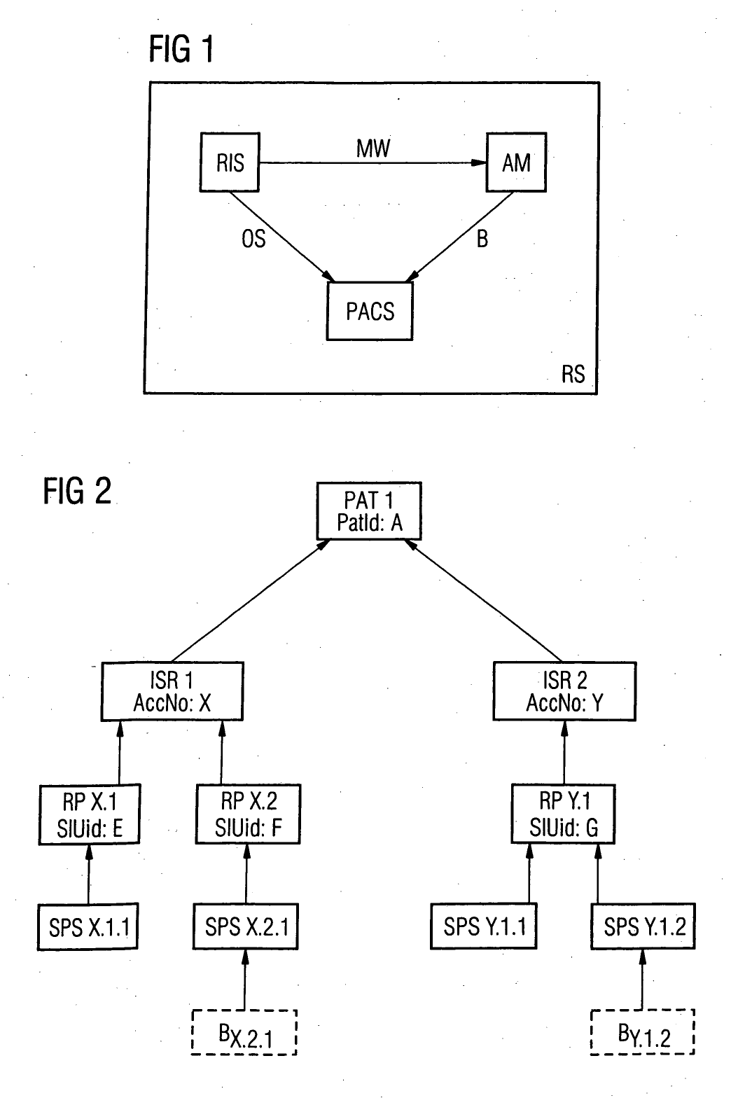

An exemplary distributed radiology system RS is shown in FIG. 1. The system includes an Order Filler RIS for planning examinations, an Acquisition Modality AM for acquisition of images B that are generated in the course of examinations, and an Image Manager PACS for administration and archiving of the images B. The three actors RIS, PACS, AM respectively form a local data set with local occurrences of data being stored such that examinations and examination results can be associated with the correct patients, and vice versa. Acquired data of the modality AM are transmitted from the Order Filler RIS using the Modality Worklists MW described above, and from the Image Manager PACS using the Order Schedules OS described above. Furthermore, images B acquired at the modality AM are transmitted from there to the Image Manager PACS.

For administration of the data, a hierarchically organized structure of associations is provided in the data set, this hierarchically organized structure of associations being realized using key data PatId, AccNo, SIUid that associate of examinations and examination results with patients, and vice versa. This hierarchical structure is exemplarily shown in FIG. 2. An examination (designated as an Imaging Service Request ISR) is accordingly associated with a patient Pat via its key PatId (also called patient number). The examination ISR is characterized by a key fashioned as an Accession Number AccNo. An examination ISR encompasses individual examination methods and procedures designated as Requested Procedure RP and characterized by a key (fashioned as a Study Instance UID SIUid) that represent individual examination steps that are designated as Scheduled Procedure Steps SPS. The examination procedures RP are associated with the examinations ISR via their key AccNo and the examination steps SPS are associated with the examination procedures RP via their key SIUid. The examination results (such as, for example, x-ray images) are arranged on the leaves of this hierarchical data structure (which is arrayed like a tree) and are associated with the examination steps. By means of this structure, each examination result of an examination step is characterized by a data tuple {PatId; AccNo; SIUid}.

This data tuple is determined in the transmitting actor given transmission of an examination result and is sent to the receiving actor together with the examination result. There the received tuple is analyzed and searched for in the local data set. This search can fail. This can have two causes: 1) The received examination result is not yet known at the receiver. It can then merely be inserted into the local data set for the first time in a known manner; 2) The received data tuple is in contradiction to the local data set and cannot be inserted into this in a known manner. In this case, a data inconsistency according to the invention exists.

The data inconsistency can have different causes. Dependent on the cause, a correction procedure matching this cause is therefore to be selected for synchronization of the inconsistent data set, which correction procedure is, for example, fashioned as a replacement CHANGE, shift MOVE and/or as a merge MERGE. Corrected data are initially, inventively acquired. This acquisition preferably ensues at a central actor, for example the Order Filler RIS. At least the corrected data are subsequently communicated to the actors affected by the data inconsistency. The communication is advantageously effected using standardized HL7 transactions that, if applicable, include a special field in which the corrected data are stored. For example, these transactions exhibit the following syntax:

MSH, PID, ZPA

The MSH and PID segments are described in the standard HL7. The segment ZPA (Patient Administration Request) is defined, for example, as follows:

| Seq | Name | DT | Len | Opt | Rep | Min | Max | Tbl |
|---|---|---|---|---|---|---|---|---|
| ZPA-1 | Study Instance | | | C | False | 0 | 1 | |
| | UID | | | | | | | |
| | source SIUid | ST | 250 | R | | 0 | 1 | |
| | target SIUid | ST | 250 | O | | 0 | 1 | |
| ZPA-2 | Accession | | | C | False | 0 | 1 | |
| | Number | | | | | | | |
| | source AccNo | ST | 250 | R | | 0 | 1 | |
| | target AccNo | ST | 250 | O | | 0 | 1 | |

Furthermore, the following HL7 transactions can be provided in which the segment ZPA is stored:

| | |
|---|---|
| Cmd | HL7 Msg Type |
| ISR MOVE | ZPA ˆ I01 |
| ISR AccNo CHANGE | ZPA ˆ I03 |
| SR MERGE | ZPA ˆ I04 |
| Study MOVE | ZPA ˆ S01 |
| Study Instance Uid CHANGE | ZPA ˆ S03 |
| Study Merge | ZPA ˆ S04 |

The selection of the matching correction procedure(s) ensues under consideration of the corrected data. This selection is refined when the current data set is also taken into account. This selection can ensue in the central actor before communication of the corrected data or in the appertaining actors after administration of the corrected data.

How the inconsistencies forming the bases of the scenarios are corrected according to the invention is subsequently, described as an example using some selected scenarios. In particular the selection of the correction procedures is explained in detail, with emphasis on the fact that the specified correction processes do not necessarily have to be those that are described as an example. Instead, the desired correction can be effected by alternative correction procedures with correspondingly adapted parameterization.

In the selected scenarios, it is assumed that an examination result of an examination step is received that is characterized by a data tuple {PatId; AccNo; SIUid}.

1) Correct Patient/Study—wrong AccNo

The keys PatId and SIUid are found in the local data set. Both keys are associated with one another. The key AccNo is not found. This information is indicated. As a result, a corrected key AccNo is acquired. It is now checked whether the corrected key AccNo is already known.

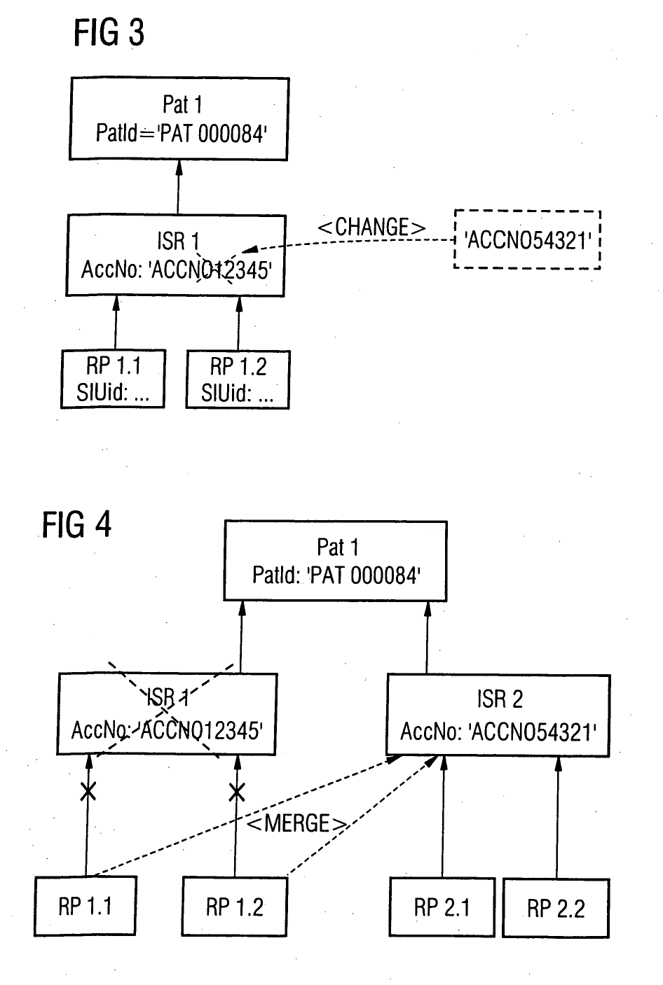

For the case that the corrected key AccNo is not yet known, a replacement CHANGE of the previous key AccNo with the corrected key AccNo is, for example, established as a correction procedure. The effects of the correction on the data set are shown in FIG. 3.

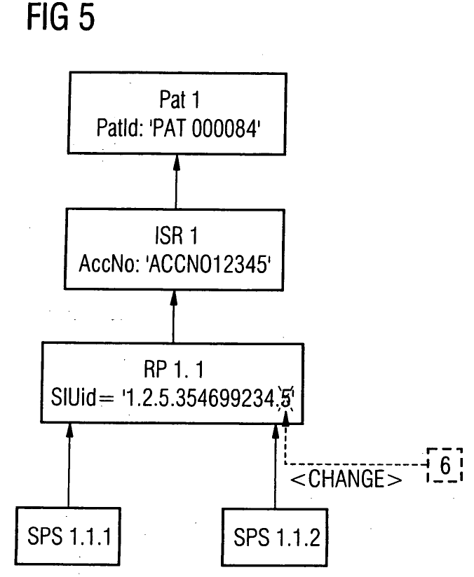

For the case that the corrected key AccNo is already known, a merging MERGE of the previous key AccNo with the known key AccNo is, for example, established as a correction procedure. The effects of the correction on the data set are shown in FIG. 4.

The associated HL7 transaction for communication of the corrected data is, for example, fashioned as follows in the case of a replacement CHANGE:

MSH|ˆ&˜|RIS||RIS|RIS|20041015142431||ZPAˆI03|247|P|2.3.1

PID|1|PAT000084||LastnameˆFirstname||19121212|M

ZPA||ACCNO12345ˆACCNO54321

The correction of the previous key AccNo ‘ACCNO12345’ to the new key AccNo ‘ACCNO54321’ is therewith communicated. The specifications regarding the patient are optional. They serve for checking whether the inconsistent examination is also still associated with the same patient after the correction. In this example, both keys AccNo should remain associated with the patient with the key PatId ‘PAT000084’. The patient's name is ‘LastnameˆFirstname’, birthdate is ‘12.12.1912’, gender is ‘M’ (=male).

2) Correct Patient/AccNo—unknown Study

The keys PatId and AccNo are found in the local data set. Both keys are associated with one another. The key SIUid is not found. This information is indicated. As a result, a corrected key SIUid is acquired. It is now checked whether the corrected key SIUid is already known.

For the case that the corrected key SIUid is not yet known, a replacement CHANGE of the previous key SIUid with the corrected key SIUid is, for example, established as a correction procedure. The effects of the correction on the data set are shown in FIG. 5.

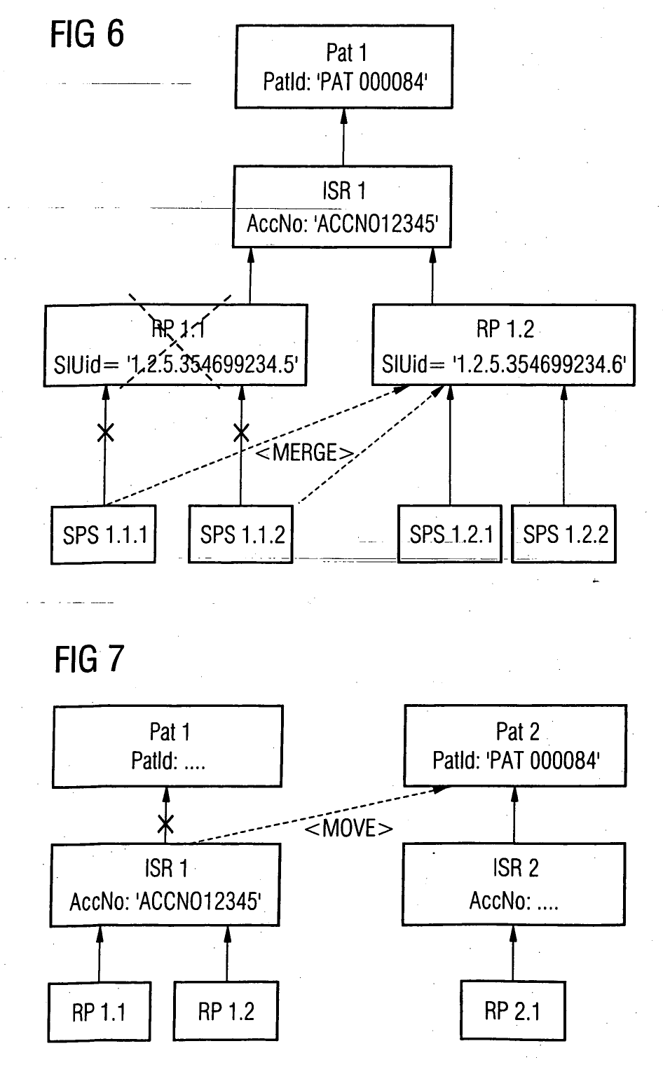

For the case that the corrected key SIUid is already known, a merging MERGE of the previous key SIUid with the known key SIUid is, for example, established as a correction procedure. The effects of the correction on the data set are shown in FIG. 6.

The associated HL7 transaction for communication of the corrected data is, for example, fashioned as follows in the case of a replacement CHANGE:

MSH|ˆ&˜\|RIS||RIS|RIS|20041015142431||ZPAˆS04|247|P|2.3.1

PID|1|PAT000084||LastnameˆFirstname||19121212|M ZPA|1.2.5.354699234.5ˆ1.2.5.354699234.6|ACCNO12345

The correction of the previous key SIUid ‘1.2.5.354699234.5’ to the new key SIUid ‘1.2.5.354699234.6’ is therewith communicated. The specifications regarding the patient are again optional and serve the same purpose as explained in the preceding. The same applies for the optional specification of the key AccNo.

3) Correct AccNo/Study—wrong Patient

The keys AccNo and SIUid are found in the local data set. Both keys are associated with one another. The key PatId is not found. This information is indicated. As a result, a corrected key PatId is acquired via selection from a list of already known keys PatId.

The corrected key PatId is already known as a result of the preceding selection procedure such that no check of the corrected key PatId is required. A shift MOVE of the examination data regarding the known key PatId is, for example, established as a correction procedure. The effects of the correction on the data set are shown in FIG. 7.

The associated HL7 transaction for communication of the corrected data is, for example, fashioned as follows in the case of a replacement MOVE:

MSH|ˆ&˜\|RIS||RIS|RIS|20041015142431||ZPAˆI01|247|P|2.3.1

PID|1|PAT000084||LastnameˆFirstname||19121212|M

ZPA||ACCNO12345

It is therewith communicated that the examinations characterized with the key AccNo ‘ACCNO12345’ should be associated with the patient characterized with the key PatId ‘PAT000084’. The further specifications regarding the patient serve for, among other things, his unambiguous identification.

4) Correct AccNo—wrong Patient/unknown Study

Only the key AccNo is found in the local data set. The keys PatId and SIUid are not found. This information is indicated. As a result, a corrected key PatId and a corrected key SIUid are acquired. The corrected key PatId is known, the corrected key SIUid is unknown.

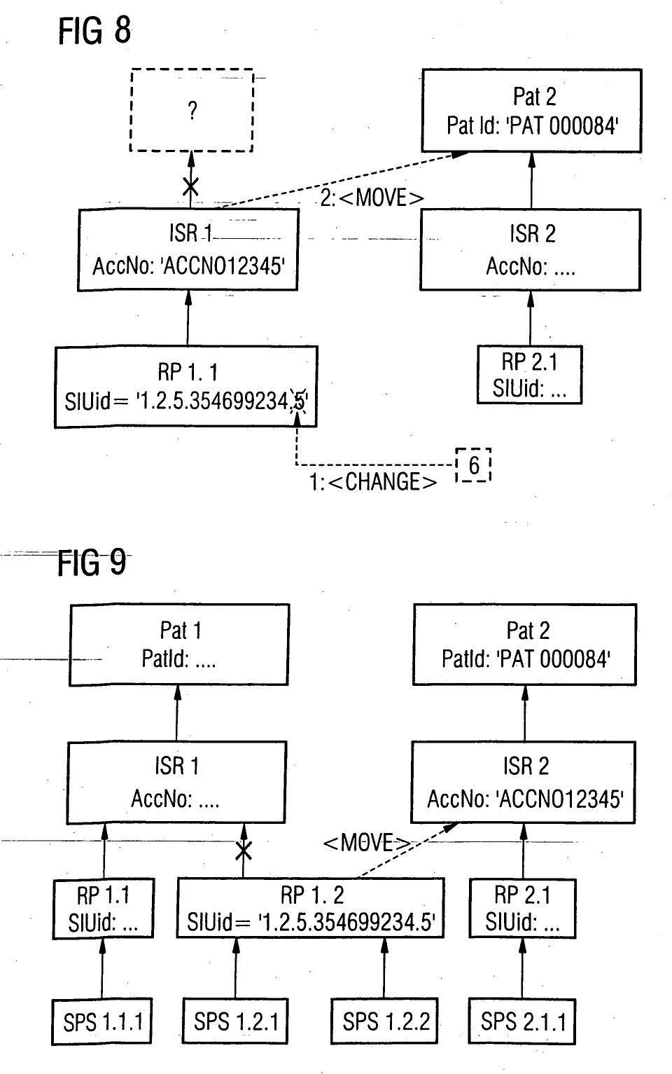

For synchronization, for example, a two-stage correction procedure is established that initially comprises a replacement CHANGE of the previous key SIUid with the corrected key SIUid and then a shift MOVE of the examination data characterized with the key AccNo to the known key PatId. The effects of the correction on the data set are shown in FIG. 8.

The associated HL7 transactions for communication of the corrected data are, for example, fashioned as follows:

MSH|ˆ&˜\|RIS||RIS|RIS|20041015142431||ZPAˆS04|247|P|2.3.1

ZPA|1.2.5.354699234.5ˆ1.2.5.354699234.6|

MSH|ˆ&˜\|RIS||RIS|RIS|20041015142431||ZPAˆI01|247|P|2.3.1

PID|1|PAT000084||LastnameˆFirstname||19121212|M

ZPA||ACCNO12345

The correction of the previous key SIUid ‘1.2.5.354699234.5’ to the new key SIUid ‘1.2.5.354699234.6’ is communicated with the first transaction. The optional specifications with regard to the patient are not specified since the check of a correct patient is not possible at this point in time as a consequence of the data inconsistency still existing at this point.

With the second transaction it is communicated that the examinations characterized with the key AccNo ‘ACCNO12345’ should be associated with the patient characterized with the key PatId ‘PAT000084’. The further specifications regarding the patient serve for, among other things, unambiguous identification thereof.

5) Correct Study—wrong Patient/AccNo

Only the key SIUid is found in the local data set. The keys PatId and SIUid [sic] are not found. This information is indicated. As a result, a corrected key AccNo is acquired. Moreover, the corrected key AccNo is already associated with the key PatId specified in the data tuple.

A one-stage correction procedure that comprises a shift MOVE of the examination procedures characterized with the key SIUid to the examination characterized with the corrected key AccNo is sufficient for synchronization. The effects of the correction on the data set are shown in FIG. 9.

The associated HL7 transactions for communication of the corrected data are, for example, fashioned as follows:

MSH|ˆ&˜\|RIS||RIS|RIS|20041015142431||ZPAˆS01|247|P|2.3.1

PID|1|PAT000084||LastnameˆFirstname||19121212|M

ZPA|1.2.5.354699234.5|ACCNO12345

With the transaction it is communicated that the examination procedures characterized with the key SIUid ‘1.2.5.354699234.5’ should be associated with the key AccNo ‘ACCNO12345’. The further specifications regarding the patient are optional and serve for checking whether the corrected examination is associated with the correct patient.

6) Wrong Patient/AccNo/Study

None of the keys SIUid, AccNo, PatId are found in the local data set. This information is indicated. A corrected key SIUid is initially acquired. The scenario is subsequently proceeded with as in the preceding scenario.

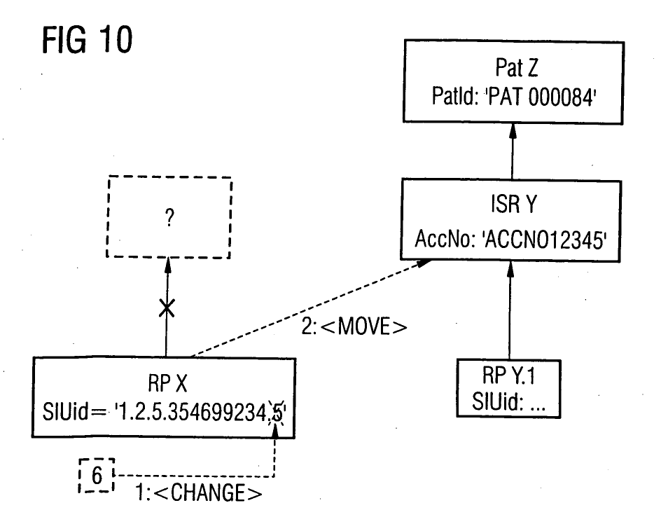

For synchronization, for example, a two-stage correction procedure is established that initially comprises a replacement CHANGE of the previous key SIUid with the corrected key SIUid and then a shift MOVE of the examination procedures characterized with the key SIUid to the examination characterized with the corrected key AccNo. The effects of the correction on the data set are shown in FIG. 10.

The associated HL7 transactions for communication of the corrected data are, for example, fashioned as follows:

MSH|ˆ&˜\|RIS||RIS|RIS|20041015142431||ZPAˆS04|247|P|2.3.1

ZPA|1.2.5.354699234.5ˆ1.2.5.354699234.6|

MSH|ˆ&˜\|RIS||RIS|RIS|20041015142431||ZPAˆS01|247|P|2.3.1

PID|1|PAT000084||LastnameˆFirstname||19121212|M

ZPA|1.2.5.354699234.6|ACCNO12345

The correction of the previous key SIUid ‘1.2.5.354699234.5’ to the new key SIUid ‘1.2.5.354699234.6’ is communicated with the first transaction. The optional specifications with regard to the patient are not specified since the check of a correct patient is not possible at this point in time as a consequence of the data inconsistency still existing at this point.

With the second transaction it is communicated that the examination procedures characterized with the key SIUid ‘1.2.5.354699234.6’ should be associated with the examination characterized with the key AccNo ACCNO12345. The optional specifications regarding the patient are now added since a check as to whether the corrected examination is associated with the correct patient is now possible.

A number of further advantages are achieved in accordance connected with the invention.

An Exception Resolution for non-associated images increases the system stability and consequently the satisfaction of customers and operating personnel since fewer images are lost as a result of wrong associations and fewer examinations must be repeated.

An implementation of the synchronization with the aid of standard-conforming extended HL7 transactions has the advantage that the existing standards can be particularly easily extended with these messages. A standard-conforming extension of the existing products is also particularly low-cost when already-existing realizations can be used again due to the conformity. Isolated applications that do not function spanning across manufacturers thus can be very efficiently avoided.

In principle a realization of the invention requires no changes of the previous prior art, but rather fundamentally a retroactive insertion as a component (in particular as a modified or additional computer program product).

The point in time of the realization is independent of the point in time of the realization of other functions.

With the invention it is ensured that the individual components of the overall system are only loaded to a slight degree, and with this the stability of the overall system is increased.

It will be apparent for those skilled in the art that the invention can be implemented distributed, partially or completely in software and on a number of physical products (particularly computer program products).

Although modifications and changes may be suggested by those skilled in the art, it is the intention of the inventor to embody within the patent warranted hereon all changes and modifications as reasonably and properly come within the scope of the inventor's contribution to the art.

I claim as my invention:

1. A method for synchronizing a distributed radiology system comprising at least two actors respectively formed by an order filler and an image manager comprising at least one of an image archive and an image acquisition modality, a plurality of local memories in which a plurality of data instances are respectively stored, each data instance representing an examination result of an examination and being stored per examination result, said data being formulated as an accession number or as a study instance, said method comprising the steps of:
automatically electronically detecting an inconsistency between data instances respectively stored in different ones of said memories;
automatically electronically displaying said inconsistency;
automatically electronically acquiring corrected data that removes said inconsistency;
automatically electronically communicating said corrected data to any of said actors affected by the inconsistency; and
automatically electronically synchronizing said distributed radiology system by correcting at least one data instance in at least one of said memories with said corrected data to remove the inconsistency from the distributed radiology system.

2. A method as claimed in claim 1 wherein at least some of said data memories are respectively associated with at least some of said actors, and wherein one of said actors transmits a data instance stored in the memory associated therewith to another memory associated with another of said actors, as a receiving actor, and wherein the step of automatically electronically detecting said inconsistency comprises automatically electronically determining when said data instance to be transmitted cannot be associated with any data instances in the memory associated with the receiving actor, before transmitting said data instance to said receiving actor.

3. A method as claimed in claim 1 wherein said actors include a central actor, and comprising automatically electronically communicating said inconsistency to said central actor, and acquiring said corrected data at said central actor.

4. A method as claimed in claim 3 comprising communicating said corrected data from said central actor to said actors affected by said inconsistency.

5. A method as claimed in claim 1 comprising communicating said corrected data as a standardized HL7 transaction.

6. A method as claimed in claim 5 comprising generating a special field in the standardized HL7 transaction and embodying said corrected data in said special field.

7. A method as claimed in claim 1 comprising correcting said local data instance by replacing said local data instance with said corrected data by a procedure selected from the group consisting of shifting to a further data instance and merging with a further data instance.

8. A computer-readable medium encoded with a computer program for synchronizing a distributed radiology system comprising at least two actors respectively formed by an order filler and an image manager comprising at least one of an image archive and an image acquisition modality, a plurality of local memories in which a plurality of data instances are respectively stored, each data instance representing an examination result of an examination and being stored per examination result, said data being formulated as an accession number or as a study instance, said medium being loadable into a computer of said system and causing said system:
to automatically electronically detect an inconsistency between data instances respectively stored in different ones of said memories;
to automatically electronically display said inconsistency;
to automatically electronically acquire corrected data that removes said inconsistency;
to automatically electronically communicate said corrected data to any of said actors affected by the inconsistency; and
to automatically electronically synchronize said distributed radiology system by correcting at least one data instance in at least one of said memories with said corrected data to remove the inconsistency from the distributed radiology system.
